(19) United States
(12) Patent Application Publication
(10) Pub. No.: US 2003/0138938 A1
(43) Pub. Date: Jul. 24, 2003

(54) Agar composition

(76) Inventors: Masaaki Kojima, Nagano (JP); Takehiko Sakai, Nagano (JP); Yuji Uzuhashi, Nagano (JP)

Correspondence Address: TREXLER, BUSHNELL, GIANGIORGI,, BLACKSTONE & MARR, LTD., 105 WEST ADAMS STREET, SUITE 3600, CHICAGO, IL, 60603, US

(21) Appl. No.: 10/318,888

(22) Filed: Dec. 13, 2002

(30) Foreign Application Priority Data
Dec. 13, 2001 (JP) 2001-380474

(51) Int. Cl. C12N 1/20; C08B 37/04

(52) U.S. Cl. 435/253.6; 536/3

(57) ABSTRACT

Agar composition is provided with excellent water retention, sufficient gelling ability and no pasty eating texture. The agar composition comprises agar having an average molecular weight of 10,000-200,000; and a thickening agent containing at least one or more of locust bean gum, tara gum, konjak mannan and cassia gum.

### CROSS-REFERENCE TO RELATED APPLICATION

This application claims foreign priority from Japanese Application No. 2001-380474, filed Dec. 13, 2002.

### BACKGROUND OF THE INVENTION

1. Field of the Invention

The present invention relates to an agar composition, which includes agar, and a thickening agent such as locust bean gum, tara gum, konjak mannan (glucomannan), cassia gum and tamarind gum.

2. Description of the Related Art

Agar is a polysaccharide obtained from red alga such as Gelidaceae and Gracilariaceae by extracting their membranal components through hot water, which are then purified and dried. It can be dissolved in hot water and cooled to form a gel. Agar has been employed in Japan until now from 350 or more years ago in various uses beginning with foods. In view of the technical background on production to obtain dried products extracted from sea algae, agar with the gel strength of 400-900 g/cm² and an average molecular weight of 200,000-400,000 is produced in general. It is desired to utilize agar in various uses more than before because it contains much dietary fibers, for example.

Agar has relatively less hydrophilic groups than other polysaccharides and retains water molecules through hydrogen bonds. Therefore, it is recognized as a fragile gel with a relatively easier syneresis. The use of agar with other thickening agents such as locust bean gum can be considered to improve its water retention. Agar consists of a neutral polysaccharide called agarose having a repetitive unit of D-galactose and 3,6-anhydro-L-galactose; and an agararopectine containing sulfate esters, pyruvic and methoxyl groups, for example, in agarose. It is a near neutral, straight chain, acidic polysaccharide with less reactive groups, and can not exhibit any reaction by interlacing of its molecules with a thickening agent such as locust bean gum. Other thickening agents prevent agar from forming its mesh structure. As a result, not only the gelling ability cannot be improved but also the syneresis can not be suppressed sufficiently. When a gel cannot be formed sufficiently, the eating texture comes pasty. Further, agar has a poor freeze-tolerance, that is, repetition of freezing and defrosting disrupts the gel tissue and invites easy syneresis. These are disadvantages.

Gelatin has been employed in the art because it has less syneresis, sufficient gelling ability, and no pasty eating texture. Gelatin is produced from glue attached to bovine bones and invites great questions and anxieties against safety raised from food, pharmaceutical and cosmetic manufacturers and consumers in view of influences from Bovine Spongiform Encephalophathy. Therefore, such a gel is desired that is an alternative of gelatin and has the same physical property.

Agar is composed of agarose and agaropectin. When it is refined elevate its agarose content as high as possible, refined agar can be obtained. Such the refined agar has been produced commercially with the same gel strength and the average molecular weight as those of the above agar. The refined agar is commercially sold as a name of agarose. The refined agar is employed as support for electrophoresis of a DNA or protein with a relatively large molecular weight as high as 100-100,000 bp. Polyacrylamide gel on the other hand is employed as support for electrophoresis of a DNA or protein with a low molecular weight of 10-1,000 bp.

The polyacrylamide gel, employed as support for electrophoresis of a DNA or protein with a low molecular weight of 10-1,000 bp, is produced by polymerizing an acrylamide with a cross linker of methylenebisacrylamide. The acrylamide is a neurotoxin and thus is not a biologically suitable substance. Accordingly, it is desired not to employ the acrylamide as support for electrophoresis of a DNA or protein with a low molecular weight of 10-1,000 bp. Rather, it is considered to employ the refined agar as the support.

However, if the refined agar is densified and used with a denser mesh of molecules to divide a low molecular DNA, it comes further turbidly white. In addition the use of the refined agar makes it difficult to decide a sequence determination and is not suitable for separation of DNA fragments with a low molecular weight of 10-1,000 bp. Specifically, although ethidium bromide is normally employed to dye a DNA, followed by irradiating from an UV lamp to cause fluorocence emission, to determine a state after electrophoresis, it is difficult to determine a clear sequence Image.

Available methods known in the art to overcome the problems on such the refined agar include: (1) a high dense use of hydroxyethylagarose developed for a low molecular DNA; (2) a mixture of γ-rays irradiated galactomannan with agarose to form a gel with an improved transparency and fine mesh structure of the gel (U.S. Pat. No. 5,230,832); and (3) the use of a combination of galactomannan with agarose in a Tris-Borate-EDTA (TBE) buffer to divide DNA fragments, Perlman et al., (Analytical Biochemistry, 163, 247-254(1987)). The method (1) has a disadvantage, however, because it includes complicated reaction steps, which require an elevated cost. The method (2) is a special method of radioactive decomposition, which has a lower resolution than that at the time when galactomannan before radioactive decomposition is employed. The method (3) has a disadvantage because a high viscosity on dissolution lowers workability and remains a poor reality.

The present invention has a first object to provide an agar composition with excellent water retention, sufficient gelling ability and no pasty eating texture.

The present invention has a second object to provide an agar composition usable as support for electrophoresis of a low molecular DNA or protein.

### SUMMARY OF THE INVENTION

To achieve the above first object, the Inventors intensively studied and consequently found that agar can exhibit reaction by interlacing of molecules with a thickening agent such as locust bean gum, konjak mannan and tara gum when the length of the molecule is adjusted, and can improve water retention and gelling ability through a synergistic effect. An agar composition according to the present invention comprises the agar having an average molecular weight of 10,000-200,000; and a thickening agent containing at least one or more of locust bean gum, tara gum, konjak mannan, cassia gum and tamarind gum.

To achieve the above second object, the inventors intensively studied and consequently found that refined agar can exhibit reaction by interlacing of molecules with a thickening agent such as locust bean gum, konjak mannan, tara gum, cassia gum and tamarind gum when the length of the molecule is adjusted, and can be applied to separate DNA fragments with a low molecular weight of 10-1,000 bp through a synergistic effect. Agar composition according to the present invention comprises refined agar having an average molecular weight of 10,000-200,000; and a thickening agent containing at least one or more of locust bean gum, konjak mannan, tara gum, cassia gum and tamarind gum. Any methods using these agar compositions can be performed in safe and low cost without any problems on workability at the time of dissolution.

### BRIEF DESCRIPTION OF THE DRAWINGS

The present invention will be more fully understood from the following detailed description with reference to the accompanying drawings, in which:

FIG. 1 is a graph showing gel strengths related to compound ratios when agars having weight average molecular weights of 10,000-200,000 are compounded with locust bean gum;

FIG. 2 is a graph showing variations in gel strength in accordance with a ratio of Agar A to a thickening agent;

FIG. 3 is a graph showing variations in gel strength in accordance with a ratio of Agar A to a thickening agent when xanthan gum is added;

FIG. 4 is a graph showing variations in rupture length in accordance with a ratio of Agar A to a thickening agent;

FIG. 5 is a graph showing variations in rupture length in accordance with a ratio of Agar A to a thickening agent when the xanthan gum is added; and

FIG. 6 is a graph showing gel strengths related to compound ratios when agars having weight average molecular weights of 10,000-200,000 are compounded with tamarind gum.

### DETAILED DESCRIPTION OF THE PREFERRED EMBODIMENTS

Preferably, the agar composition according to the present invention may be prepared in the form of a gel, and the agar and the thickening agent may have a compound ratio of 1:0.01-9.5. Preferably, the agar composition according to the present invention may further comprise the xanthan gum. Preferably, the agar and the thickening agent may be purified and transparent, and may be prepared to have a freeze-tolerance.

Preferably, the agar composition according to the present invention may have the dynamic storage viscoelasticity (G′) of 10¹-10³ Pa and loss tangent (tan δ) of 10⁻²-10⁰ in 1% density at 15° C. Preferably, it has the physical property close to that of gelatin in particular.

The refined agar in the agar composition according to the present invention is obtained by refining agar to elevate its agarose content as high as possible, for example, to contain the agarose content as close as 100%. The thickening agent may be prepared low molecular by hydrolysis.

### EXAMPLES

### Experimental Example 1

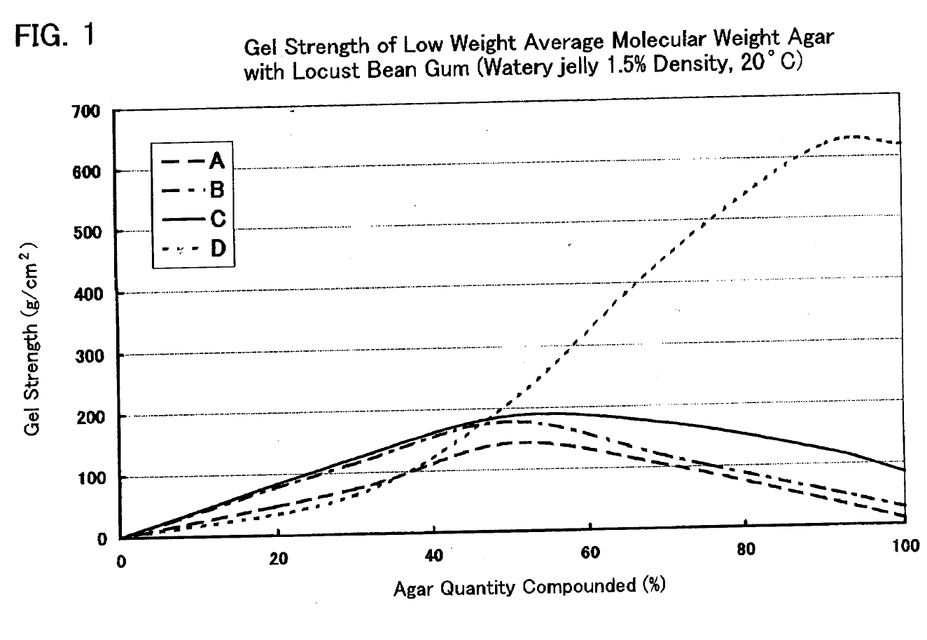

Agar A, B and C having average molecular weights shown in Table 1 are compounded with locust bean gum (available from CP Kelko Inc.) In various compound ratios to measure gel strengths (watery jelly in 1.5% density at 20° C.) using a rheometer (available from Sun Scientific Co., Ltd.). The measured results are shown in FIG. 1. The agar A, B and C were obtained by hydrolyzing normal agar D that has an average molecular weight of 200,000-400,000 (available from Ina Food Industry Co., Ltd.)

TABLE 1

| | Weight Average |
|---|---|
| | Molecular Weight |
| Agar A | 72,000 |
| Agar B | 109,000 |
| Agar C | 198,000 |

As obvious from FIG. 1, the agar A, B and C can obtain synergistic effects from the use of the locust bean gum together.

### Experimental Example 2

The agar B, the agar D, konjak mannan (available from Ina Food Industry Co., Ltd.) and tara gum (available from Ina Food Industry Co., Ltd.) were compounded in the compound ratios shown in Table 2 to obtain agar compositions according to Examples 1 and 2 and agar compositions of Comparative Examples 1-4.

TABLE 2

| | | | Konjak | Tara | Xanthan |
|---|---|---|---|---|---|
| | Agar B | Agar D | Mannan | Gum | Gum |
| Example 1 | 50 | | 50 | | |
| Example 2 | 50 | | | 50 | |
| Example 3 | 50 | | 40 | | 10 |
| Comparative | 100 | | | | |
| Example 1 | | | | | |
| Comparative | | 100 | | | |
| Example 2 | | | | | |
| Comparative | | 50 | 50 | | |
| Example 3 | | | | | |
| Comparative | | 50 | | 50 | |
| Example 4 | | | | | |

The agar compositions according to Examples 1 and 2 and Comparative Examples 1-4 are subjected to measurements of gel strength (watery jelly in 1.5% density at 20° C.) using a rheometer (available from Sun Scientific Co., Ltd.), of which results are shown in Table 3. As obvious from Table 3, it is found that the agar compositions according to Examples 1 and 2 have larger gel strengths than that of the agar B according to Comparative Example 1 due to the synergistic effect from the use of the konjak mannan or the tara gum together. In contrast, the agar compositions according to Comparative Examples 3 and 4 have smaller gel strengths than that of the agar D according to Comparative Example 2.

TABLE 3

| | 1.5% Gel Strength | Rupture |
|---|---|---|
| | (g/cm²) | Distance (mm) |
| Example 1 | 74 | 15.2 |
| Example 2 | 59 | |
| Example 3 | 124 | 20.6 |
| Comparative | 30 | |
| Example 1 | | |
| Comparative | 619 | |
| Example 2 | | |
| Comparative | 207 | |
| Example 3 | | |
| Comparative | 168 | |
| Example 4 | | |

### Experimental Example 3

Xanthan gum (available from CP Kelko Co., Ltd.) is additionally compounded in the agar composition according to Example 1 to obtain agar composition as Example 3 having a compound ratio shown in Table 2. The agar composition according to Example 3 is subjected to a measurement of gel strength (watery jelly in 1.5% density at 20° C.) using a rheometer (available from Sun Scientific Co., Ltd.), of which result is shown in Table 3. As obvious from Table 3, it is found that the agar composition with the use of the xanthan gum together according to Example 3 has an increased gel strength compared to that of the agar composition according to Example 1. The agar compositions according to Examples 1 and 3 are subjected to measurements of rupture distance using a rheometer (available from Sun Scientific Co., Ltd.), of which results are shown in Table 3. As obvious from Table 3, it is found that the agar composition with the use of the xanthan gum together according to Example 3 has an elongated rupture distance and an increased gel viscoelasticity compared to those of the agar composition according to Example 1.

### Experimental Example 4

The agar B, the agar D, or gelatin (200 bloom, alkali-treated gelatin, available from Nitta Gelatin Inc.), locust bean gum (available from CP Kelko Co.. Ltd.), xanthan gum (available from CP Kelko Co., Ltd.) and consommé soup are compounded in compound ratios shown in Table 4 to obtain aspic jellies as Example 4 and Comparative Examples 5 and 6.

TABLE 4

| | | Comparative | Comparative |
|---|---|---|---|
| | Example 4 | Example 5 | Example 6 |
| Agar B | 2 parts | | |
| Agar D | | 2 parts | |
| Gelatin | | | 30 parts |
| Locust Bean Gum | 7 parts | 7 parts | |
| Xanthan Gum | 1 part | 1 part | |
| Corn Soup | 990 parts | 990 parts | 970 parts |

The agar B, the agar D, or gelatin (200 bloom, alkali-treated gelatin, available from Nitta Gelatin Inc.), locust bean gum (available from CP Kelko Co., Ltd.), xanthan gum (available from CP Kelko Co., Ltd.), sugar, grape juice (⅕ condensed, available from Alps Co., Ltd.) and water are compounded in compound ratios shown in Table 5 to obtain grape jellies as Example 5 and Comparative Examples 7 and 8.

TABLE 5

| | | Comparative | Comparative |
|---|---|---|---|
| | Example 5 | Example 7 | Example 8 |
| Agar B | 1.4 parts | | |
| Agar D | | 1.4 parts | |
| Gelatin | | | 25 parts |
| Locust Bean Gum | 4.9 parts | 4.9 parts | |
| Xanthan Gum | 0.7 part | 0.7 part | |
| Sugar | 100 parts | 100 parts | 100 parts |
| Grape Juice | 20 parts | 20 parts | 20 parts |
| Water | 873 parts | 873 parts | 855 parts |

The jellies according to Examples 4 and 5 and Comparative Examples 5-8 were subjected to measurements of dynamic viscoelasticity using an Ares dynamic viscoelasticity meter (available from Rheometric Inc.). As a result, Example 4 exhibits a gel physical property analogous to that of Comparative Example 6, and Example 5 to that of Comparative Example 8. The jellies according to Examples 4 and 5 and Comparative Examples 5-8 were also subjected to evaluations of eating texture by panelists. As a result, Example 4 exhibits eating texture close to that of Comparative Example 6, and Example 5 to that of Comparative Example 8, though they are different in melting in mouths. The jellies according to Comparative Examples 5 and 7 obtain such an evaluation that they are strongly pasty, though they are different in hardness from those according to Examples 4 and 5. In a word, it is found that the jellies according to Examples 4 and 5 have a gel physical property and eating texture close to that of the jelly obtained from the gelatin.

The jellies according to Examples 4 and 5 and Comparative Examples 5 and 7 were slowly frozen for a day in a refrigerator at −20° C. and then naturally defrosted to test restoration of the jelly. The jellies according to Examples 4 and 5 were almost restored. Although the jellies according to Comparative Examples 5 and 7 were apparently restored, they had denatured tissues, which were determined from a cross sectional view. A syneresis quantity in 100 g after frozen and defrosted was measured. The measured results are shown in Table 6. As shown in Table 6, it is found obviously that the gels of Examples 4 and 5 have less syneresis quantities and rich freeze-tolerances.

TABLE 6

| | Syneresis Quantity (mg) |
|---|---|
| Example 4 | 683 |
| Example 5 | 1033 |
| Comparative Example 5 | 3050 |
| Comparative Example 7 | 3433 |

### Experimental Example 5

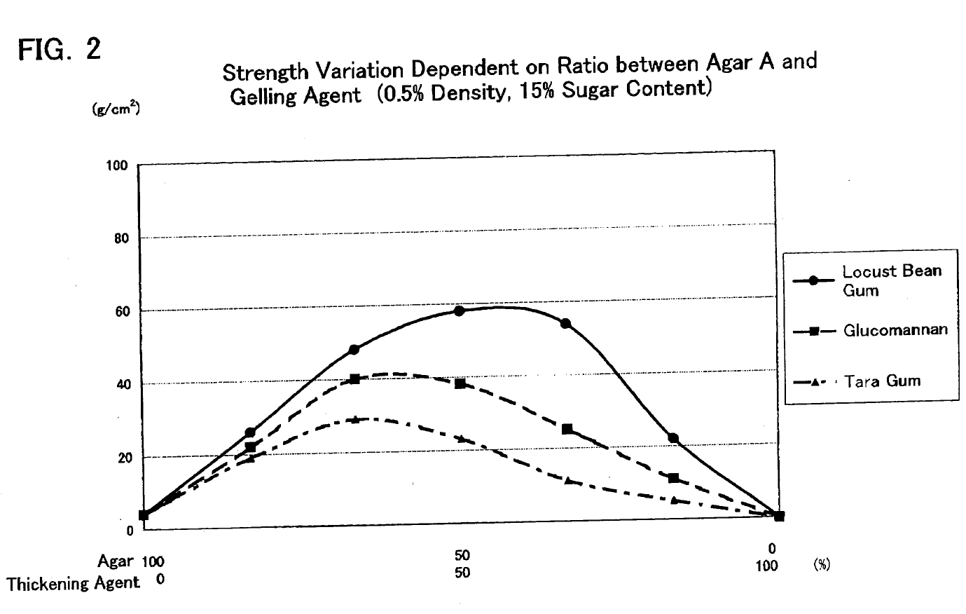

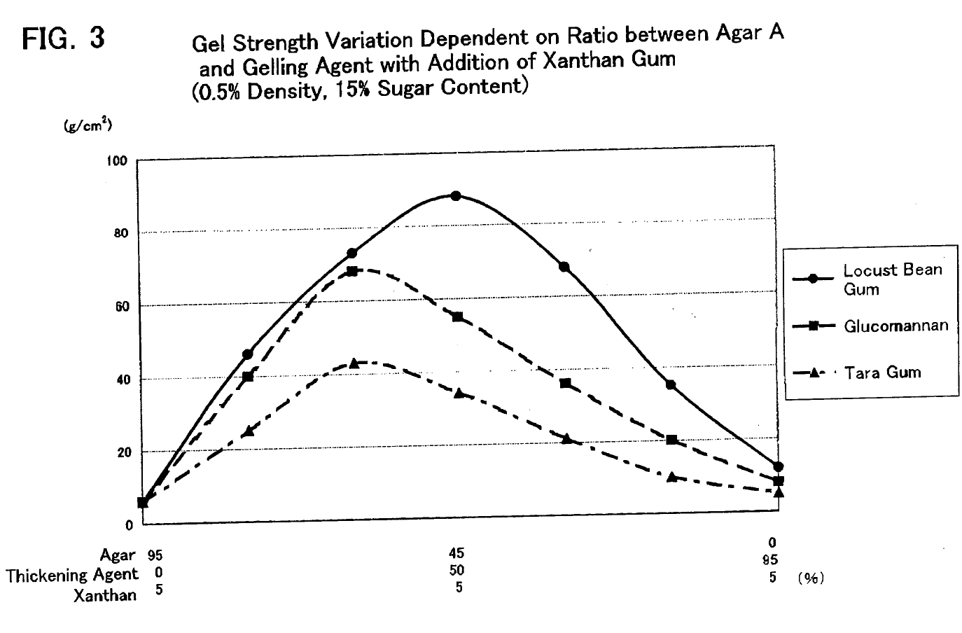

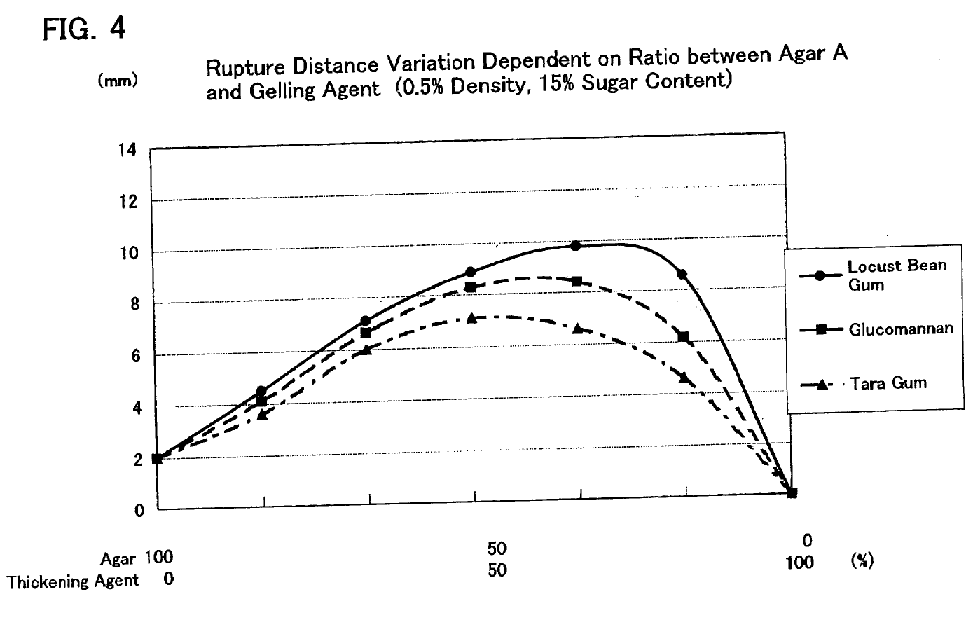

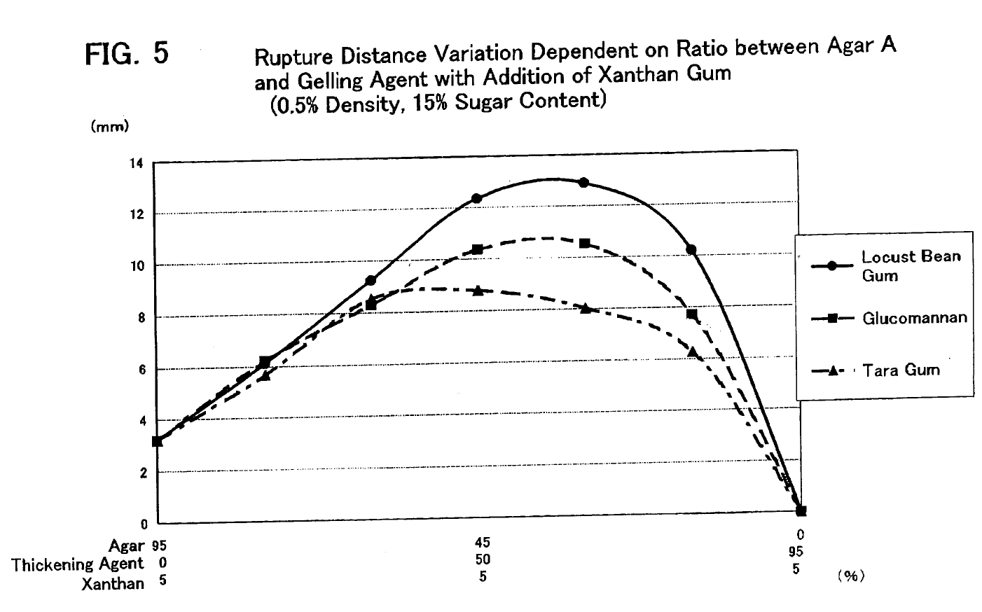

The agar A, a thickening agent (locust bean gum from CP Kelko Co., Ltd.), tara gum (from Ina Food Industry Co., Ltd.) and konjak mannan (from Ina Food Industry Co., Ltd.) were compounded in a compound ratio shown in Table 7 to obtain an agar composition. The agar A, the locust bean gum (from CP Kelko Co., Ltd.), the tara gum (from Ina Food Industry Co., Ltd.), the konjak mannan (from Ina Food Industry Co., Ltd.) and the xanthan gum (from CP Kelko Co., Ltd.) as a thickening agent were compounded in a compound ratio shown in Table 8 to obtain another agar composition. These agar compositions were subjected to measurements of gel strength (watery jelly in 1.5% density at 20° C. with 15% sugar content of sucrose) and rupture distance using rheometer (from Sun Scientific Co. Ltd.). The measured results are shown in FIGS. 2-5.

TABLE 7

| | Agar A | | | | | |
|---|---|---|---|---|---|---|
| | 0 | 10 | 30 | 50 | 70 | 100 |
| Thickening | 100 | 90 | 70 | 50 | 30 | 0 |
| agent | | | | | | |
| | | | | | | (%) |

TABLE B

Agar A

| | 0 | 10 | 30 | 50 | 70 | 90 | 95 |
|---|---|---|---|---|---|---|---|
| Thickening | 95 | 85 | 65 | 45 | 25 | 5 | 0 |
| agent | | | | | | | |
| Xanthan | 5 | 5 | 5 | 5 | 5 | 5 | 5 |
| Gum | | | | | | | |
| | | | | | | | (%) |

As shown in FIGS. 2-5, it is found that addition of the xanthan gum provides the agar composition with an elongated rupture distance and an increased gel viscoelasticity.

### Experimental Example 6

The agar A, the gelatin (200 bloom, alkali-treated gelatin from Nitta Gelatin Inc.), the locust bean gum (from CP Kelko Co., Ltd.), and the xanthan gum (from CP Kelko Co., Ltd.) were compounded in a compound ratio shown in Table 9 to obtain jellies with eating texture of gummy according to Example 6 and Comparative Example 9.

TABLE 9

| | | Comparative |
|---|---|---|
| | Example 6 | Example 9 |
| Agar A | 0.6 | |
| Locust Bean Gum | 1.9 | |
| Xanthan Gum | 0.3 | |
| Gelatin | | 8 |
| 1/5 Condensed Juice | 2 | 2 |
| Sugar | 37 | 37 |
| Millet Jelly | 25 | 25 |
| Sorbitol | 25 | 25 |
| Citric Acid | 0.6 | 0.6 |
| Sodium Citrate | 0.4 | 0.4 |
| Spices | Proper Quantity | Proper Quantity |
| Water | 10 | 10 |
| | | (Parts) |

These gummy jellies according to Example 6 and Comparative Example 9 were subjected to measurements of gel strength and rupture distance using rheometer (from Sun Scientific Co., Ltd.). The measured results are shown in Table 10.

TABLE 10

| | | Comparative |
|---|---|---|
| | Example 6 | Example 9 |
| Gel Strength (g) | 240 | 245 |
| Rupture Distance | 4.8 | 4.7 |
| (mm) | | |

As obvious from Table 10, the gummy jelly according to Example 6 exhibits an almost same value as that of the conventional gummy jelly according to Comparative Example 9. The gummy jellies according to Example 6 and Comparative Example 9 were subjected to a functional test, resulting in that the gummy jelly according to Example 6 exhibits an almost same eating texture as that of the conventional gummy jelly according to Comparative Example 9.

### Experimental Example 7

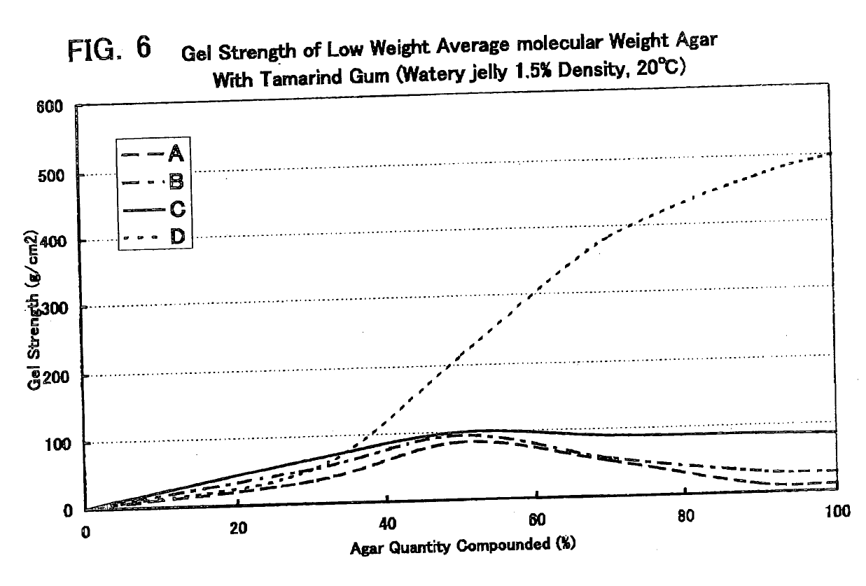

Agar A, B and C having average molecular weights shown in Table 1 are compounded with tamarind gum (available from Dainippon Pharmaceutical Co., LTD) in various compound ratios to measure gel strengths (watery jelly in 1.5% density at 20° C.) using a rheometer (available from Sun Scientific Co., Ltd.). The measured results are shown in FIG. 6.

As obvious from FIG. 6, the agar A, B and C can obtain synergistic effects from the use of the tamarind gum together.

### Experimental Example 8

Refined agar Z with an average molecular weight of 230,000 (available from Wako Pure Chemical Industries, Ltd.) was hydrolyzed with an acid to obtain refined agar X and Y with average molecular weights indicated in Table 11. These refined agar X, Y, Z were subjected to measurements of gel strength (watery jelly in 1.5% density at 20° C.) using a rheometer (available from Sun Scientific Co., Ltd.). The measured results are shown in Table 11.

TABLE 11

| | Average Molecular | |
|---|---|---|
| | Weight | Gel Strength |
| Refined agar X | 180,000 | 600 |
| Refined agar Y | 140,000 | 300 |
| Refined agar Z | 230,000 | 1,200 |

These refined agar X, Y, Z were then mixed with locust bean gum (available from CP Kelko Co., Ltd.) or tamarind gum, which is prepared by hydrolyzing with tamarind gun with an average molecular weight of 650,000 (available from Ina Food Industry Co., Ltd.) to have an average molecular weight of 320,000, at ratios indicated in Table 12 to obtain agar compositions according to Examples 7-10 and Comparative Examples 10 and 11.

TABLE 12

| | | | | Locust | | |
|---|---|---|---|---|---|---|
| | Refined | Refined | Refined | Bean | Tamarind | Buffer |
| | agar X | agar Y | agar Z | Gum | Gum | Solution |
| Example 7 | 2.0 | | | 1.0 | | 97.0 |
| Example 8 | | 2.0 | | 1.0 | | 97.0 |
| Example 9 | 1.5 | | | | 2.0 | 96.5 |
| Exomple 10 | | 1.5 | | | 2.0 | 96.5 |
| Comparative | | | 2.0 | 1.0 | | 97.0 |
| Example 10 | | | | | | |
| Comparative | | | 1.5 | | 2.0 | 96.5 |
| Example 11 | | | | | | |
| | | | | | | (%) |

These agar compositions according to Examples 7-10 and Comparative Examples 10 and 11 were subjected to measurements of viscosity on dissolution and gel strength. The viscosity on dissolution was measured using a B-type viscosimeter (available from Shibaura Systems Co., Ltd.) at 80° C. of measuring temperature and the gel strength using a Nikkan-Suishiki strength meter at 20° C. of measuring temperature. These results are shown in Table 13.

TABLE 13

| | Viscosity | |
|---|---|---|
| | on Dissolution | Gel Strength |
| Example 7 | 320 | 690 |
| Example 8 | 190 | 550 |
| Example 9 | 80 | 860 |
| Example 10 | 50 | 570 |
| Comparative | 650 | 780 |
| Example 10 | | |
| Comparative | 120 | 480 |
| Example 11 | | |

15% polyacrylamide is produced as Comparative Example 12. The agar compositions according to Examples 7-10, the agar compositions according to Comparative Examples 10 and 11, and the polyacrylamide according to Comparative Example 12 were employed as support for electrophoresis of a low molecular DNA under the following condition.

Migration Condition

| | |
|---|---|
| Migration DNA: | pUC19/MspI digest |
| | φX174/HaeIII digest |
| Buffer: | 10 mmol/l Tris/Hcl (pH 7.9) |
| | 1 mmol/l EDTA, 20 mmol/l NaCl |
| Migration Time: | 100 V, 5 Hours |

The above results indicate that the agar compositions according to Comparative Examples 10 and 11 exhibit high viscosity on dissolution and thus poor workability. In contrast, the agar compositions according to Examples 7-10 exhibit relatively lower viscosity on dissolution than those of Comparative Examples with no problem on workability, and exhibit the same resolution as that of polyacrylamide. In addition, it is confirmed that the agar compositions according to Examples 7-10 have superior transparence and a sequence image of DNA is clear.

As obvious from the forgoing, according to the present invention, when the length of agar molecule is adjusted, the agar can exhibit reaction by interlacing of molecules with a thickening agent such as locust bean gum, konjak mannan and tara gum, and can improve water retention and gelling ability through a synergistic effect.

As described above, Agar composition according to the present invention comprises refined agar having an average molecular weight of 10,000-200,000; and a thickening agent containing at least one or more of locust bean gum, konjak mannan, tara gum, cassia gum and tamarind gum. Therefore, it is possible to provide agar composition usable as support for electrophoresis of a low molecular DNA or protein.

Having described the embodiments consistent with the invention, other embodiments and variations consistent with the invention will be apparent to those skilled in the art. Therefore, the invention should not be viewed as limited to the disclosed embodiments but rather should be viewed as limited only by the spirit and scope of the appended claims.

What is claimed is:

1. Agar composition, comprising:
agar having an average molecular weight of 10,000-200,000; and
a thickening agent containing at least one or more of locust bean gum, tara gum, konjak mannan, cassia gum and tamarind gum.

2. The agar composition according to claim 1, wherein said composition is prepared in the form of a gel.

3. The agar composition according to claim 1, wherein said agar and said thickening agent have a compound ratio of 1:0.01-9.5.

4. The agar composition according to claim 1, further comprising xanthan gum.

5. The agar composition according to claim 1, said composition is prepared to have a freeze-tolerance.

6. The agar composition according to claim 1, said composition has a dynamic storage viscoelasticity (G′) of 10¹-10³ Pa and a loss tangent (tan δ) of 10⁻²-10⁰ in 1% density at 15° C.

7. Agar composition, comprising:
refined agar having an average molecular weight of 10,000-200,000; and
a thickening agent containing at least one or more of locust bean gum, tara gum, konjak mannan, cassia gum and tamarind gum.

8. The agar composition according to claim 7 wherein said composition is prepared in the form of a gel.

9. The agar composition according to claim 7, wherein said refined agar and said thickening agent have a compound ratio of 1:0.01-9.5.
